(19) United States
(12) Patent Application Publication
(10) Pub. No.: US 2019/0200919 A1
(43) Pub. Date: Jul. 4, 2019

(54) POSTURE ANALYSIS DEVICE, POSTURE ANALYSIS METHOD, AND COMPUTER-READABLE RECORDING MEDIUM

(71) Applicant: NEC Solution Innovators, Ltd., Koto-ku, Tokyo (JP)

(72) Inventor: Yusuke NAKAO, Tokyo (JP)

(73) Assignee: NEC Solution Innovators, Ltd., Koto-ku, Tokyo (JP)

(21) Appl. No.: 16/311,814

(22) PCT Filed: Jun. 23, 2017
(86) PCT No.: PCT/JP2017/023310

§ 371 Date: Dec. 20, 2018

(30) Foreign Application Priority Data
Jun. 23, 2016 (JP) 2016-124876

(51) Int. Cl. A61B 5/00; G06T 7/521; A61B 5/11; G06K 9/00

(52) U.S. Cl. CPC A61B 5/4561; G06T 7/521; A61B 5/1128; G16H 50/30; A61B 5/1116; G06K 9/00369; A61B 5/1114

(57) ABSTRACT

A posture analysis device (10) is a device for analyzing the posture of a subject, and includes a data acquisition unit (11) configured to acquire image data to which the depth of each pixel is added, from a depth sensor that is disposed to capture an image of the subject, a skeleton information creation unit (12) configured to create skeleton information for specifying positions of a plurality of sites of the subject based on the image data, a state specifying unit (13) configured to specify states of the back, the upper limbs, and the lower limbs of the subject based on the skeleton information, and a posture analysis unit (14) configured to analyze the posture of the subject based on the specified states of the back, the upper limbs, and the lower limbs of the subject.

### TECHNICAL FIELD

The present invention relates to a posture analysis device and a posture analysis method for analyzing the posture of a person, and further relates to a computer-readable recording medium in which a program for realizing the device and method is recorded.

### BACKGROUND ART

Conventionally, health disorders such lumbago sometimes occur at production sites, construction sites, and the like due to workers taking an unnatural posture or the like. Also, caregivers have similar health disorders at nursing facilities, hospitals, and the like. Thus, suppression of the occurrence of health disorders such as lumbago by analyzing the posture of a worker, a caregiver, or the like is required.

Specifically, OWAS (Ovako Working Posture Analysing System) is known as a method for analyzing posture (see Non-Patent Documents 1 and 2). Here, OWAS will be described with reference to FIGS. 8 and 9. FIG. 8 is a diagram showing posture codes used in OWAS. FIG. 9 is a diagram showing an evaluation table used in OWAS.

As shown in FIG. 8, the posture codes are set for each of the back, the upper limbs, the lower limbs, and the weight of an object. Also, the contents of each posture code shown in FIG. 8 are as follows.

Back

1: Back is straight

2: Bent forward or backward

3: Twisted or side of body bent

4: Twisting motion and bent forward/backward or bent side of body

Upper Limbs

1: Both arms are below shoulders

2: One arm is at shoulder height or higher

3: Both arms are at shoulder height or higher

Lower Limbs

1: Seated

2: Standing upright

3: Weight on one leg (leg that weight is on is straight)

4: Semi-crouching

5: Semi-crouching with weight on one leg

6: Kneeling on both knees or one knee

7: Walking (moving)

Weight

1:10 kg or less

2: 10 to 20 kg

3: Over 20 kg

First, an analyst collates, for each worker, the movement of the back, the upper limbs, and the lower limbs with the posture codes shown in FIG. 8 for each motion while observing the working situation of the worker that was shot on video. Then, the analyst specifies codes corresponding to the back, the upper limbs, and the lower limbs, and records the specified codes. Also, the analyst records the codes corresponding to the weight of objects handled by the worker. Thereafter, the analyst applies the recorded codes to the evaluation table shown in FIG. 9, and determines the risks of health disorders for each task.

In FIG. 9, numerical values other than the codes express risks. The contents of specific risks are as follows.

1: Musculoskeletal load caused by this posture is not a problem. Risk is extremely low.

2: This posture is detrimental to the musculoskeletal system. Risk is low but needs to be remedied soon.

3: This posture is detrimental to musculoskeletal system. Risk is high and should be remedied urgently.

4: This posture is very detrimental to musculoskeletal system. Risk is extremely high and should be remedied immediately.

In this manner, use of OWAS makes it possible to objectively evaluate the load on workers, caregivers, and the like. As a result, working processes and the like can be easily reviewed, and the occurrence of health disorders is suppressed in various fields such as production, construction, nursing care, medical care, and the like.

### LIST OF PRIOR ART DOCUMENTS

### Non Patent Document

Non-Patent Document 1: “Outline of ‘Guidelines on the Prevention of Lumbago in the Workplace’ and Examples of Risk Evaluation Method for Prevention of Lumbago and like”, [online], Aichi Labor Bureau, Retrieved on Jun. 1, 2014, Internet <URL: http://aichi-roudoukyoku.jsite.mhlw.go.jp/library/aichi-roudoukyoku/jyoho/roudoueisei/youtuubousi.pdf>
Non-Patent Document 2: ““OWAS: Ovako Working Posture Analysing System”, [online], Jun. 1, 2014, Aichi Labor Bureau, Retrieved on Jun. 1, 2014, Internet <URL: http://aichi-roudoukyoku.jsite.mhlw.go.jp/library/aichi-roudoukyoku/jyoho/roudoueisei/youtuubousi.pdf>

### DISCLOSURE OF THE INVENTION

### Problems to be Solved by the Invention

Incidentally, as described above, in general, OWAS is performed through manual video analysis. Thus, it is problematic in that execution of OWAS needs excessive time and effort. Also, computer software for supporting execution of OWAS has been developed, but even when this software is utilized, the task of specifying the codes based on the working situation needs be performed manually, and there is a limit to the reduction of time and effort.

An example of an object of the present invention is to solve the above-described problems, and provide a posture analysis device, a posture analysis method, and a computer-readable recording medium that can analyze the posture of a subject without performing manual tasks.

### Means for Solving the Problems

In order to achieve the above-described object, a posture analysis device in an aspect of the present invention is a device for analyzing a posture of a subject, the device including:

a data acquisition unit configured to acquire data that varies according to a motion of the subject;

a skeleton information creation unit configured to create skeleton information for specifying positions of a plurality of sites of the subject based on the data;

a state specifying unit configured to specify states of a back, upper limbs, and lower limbs of the subject based on the skeleton information; and

a posture analysis unit configured to analyze the posture of the subject based on the specified states of the back, the upper limbs, and the lower limbs of the subject.

Also, in order to achieve the above-described object, a posture analysis method in an aspect of the present invention is a method for analyzing a posture of a subject, the method including:

(a) a step of acquiring data that varies according to a motion of the subject;

(b) a step of creating skeleton information for specifying positions of a plurality of sites of the subject based on the data;

(c) a step of specifying states of a back, upper limbs, and lower limbs of the subject based on the skeleton information; and

(d) a step of analyzing the posture of the subject based on the specified states of the back, the upper limbs, and the lower limbs of the subject.

Furthermore, in order to achieve the above-described object, a computer-readable recording medium in an aspect of the present invention is a computer-readable recording medium in which a program for analyzing a posture of a subject by a computer is recorded, the program including commands for causing the computer to execute:

(a) a step of acquiring data that varies according to a motion of the subject;

(b) a step of creating skeleton information for specifying positions of a plurality of sites of the subject based on the data;

(c) a step of specifying states of a back, upper limbs, and lower limbs of the subject based on the skeleton information; and

(d) a step of analyzing the posture of the subject based on the specified states of the back, the upper limbs, and the lower limbs of the subject.

### Advantageous Effects of the Invention

As described above, according to the present invention, it is possible to analyze the posture of a subject without performing manual tasks.

### BRIEF DESCRIPTION OF THE DRAWINGS

FIG. 1 is a block diagram showing a schematic configuration of a posture analysis device in an embodiment of the present invention.

FIG. 2 is a block diagram showing a specific configuration of the posture analysis device this embodiment.

FIG. 3 is a diagram showing an example of skeleton information created in an embodiment of the present invention.

FIG. 4 are diagrams illustrating three-dimensional coordinate calculation processing in an embodiment of the present invention, with FIG. 4(a) showing calculation processing in a horizontal direction (X-coordinate) of an image and FIG. 4(b) showing calculation processing in a vertical direction (Y-coordinate) of an image.

FIG. 5 is a flowchart showing operations of the posture analysis device in an embodiment of the present invention.

FIG. 6 is a flowchart that specifically shows lower limb code determination processing shown in FIG. 5.

FIG. 7 is a block diagram showing an example of a computer for realizing the posture analysis device in an embodiment of the present invention.

FIG. 8 is a diagram showing posture codes used in OWAS.

FIG. 9 is a diagram showing an evaluation table used in OWAS.

### MODE FOR CARRYING OUT THE INVENTION

### Embodiment

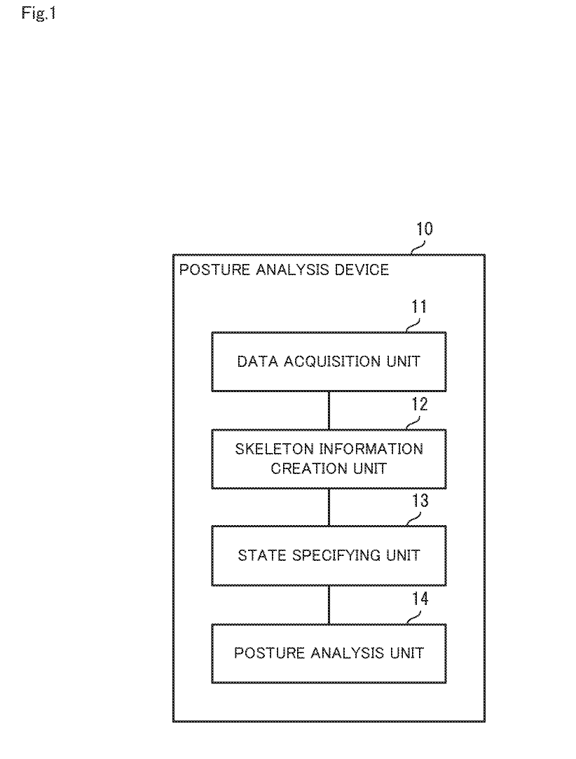

Hereinafter, a posture analysis device, a posture analysis method, and a program in an embodiment of the present invention will be described with reference to FIGS. 1 to 7.

Device Configuration

First, a schematic configuration of the posture analysis device in the present embodiment will be described with reference to FIG. 1. FIG. 1 is a block diagram showing a schematic configuration of a posture analysis device in an embodiment of the present invention.

A posture analysis device 10 in the present embodiment shown in FIG. 1 is a device for analyzing the posture of a subject. As shown in FIG. 1, the posture analysis device 10 includes a data acquisition unit 11, a skeleton information creation unit 12, a state specifying unit 13, and a posture analysis unit 14.

The data acquisition unit 11 acquires data that varies according to the motion of the subject. The skeleton information creation unit 12 creates skeleton information for specifying positions of a plurality of sites of the subject based on the acquired data.

The state specifying unit 13 specifies states of the back, the upper limbs, and the lower limbs of the subject based on the skeleton information. The posture analysis unit 14 analyzes the posture of the subject based on the specified states of the back, the upper limbs, and the lower limbs of the subject.

In this manner, in the present embodiment, it is possible to specify the postures of workers, caregivers, and the like from data that varies according to the motion of the subject. That is, according to the present embodiment, it is possible to analyze the posture of a subject without performing manual tasks.

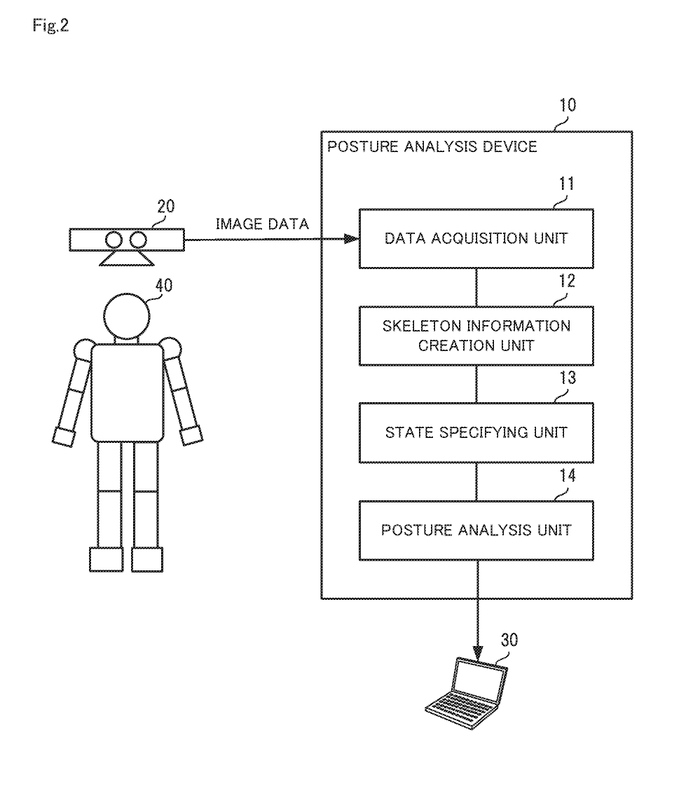

Subsequently, a specific configuration of the posture analysis device 10 in the present embodiment will be described with reference to FIG. 2. FIG. 2 is a block diagram showing a specific configuration of the posture analysis device in the present embodiment.

As shown in FIG. 2, a depth sensor 20 and a terminal device 30 of an analyst are connected to the posture analysis device 10 in the present embodiment. The depth sensor 20 includes a light source that emits infrared laser light in a specific pattern and an image sensor that receives infrared rays reflected by an object, and outputs image data to which the depth of each pixel is added, using the light source and image sensor. A specific example of the depth sensor is an existing depth sensor such as a Kinect (registered trademark) depth sensor.

Also, the depth sensor 20 is disposed so as to be capable of capturing the motion of a subject 40. Thus, in the present embodiment, the data acquisition unit 11 acquires, from the depth sensor 20, image data with the depth at which the subject 40 appears, as data that varies according to the motion of the subject 40, and inputs this data to the skeleton information creation unit 12.

In the present embodiment, the skeleton information creation unit 12 calculates three-dimensional coordinates of a specific site of a user using coordinates on the image data and the depth added to pixels for every image data, and creates skeleton information using the calculated three-dimensional coordinates.

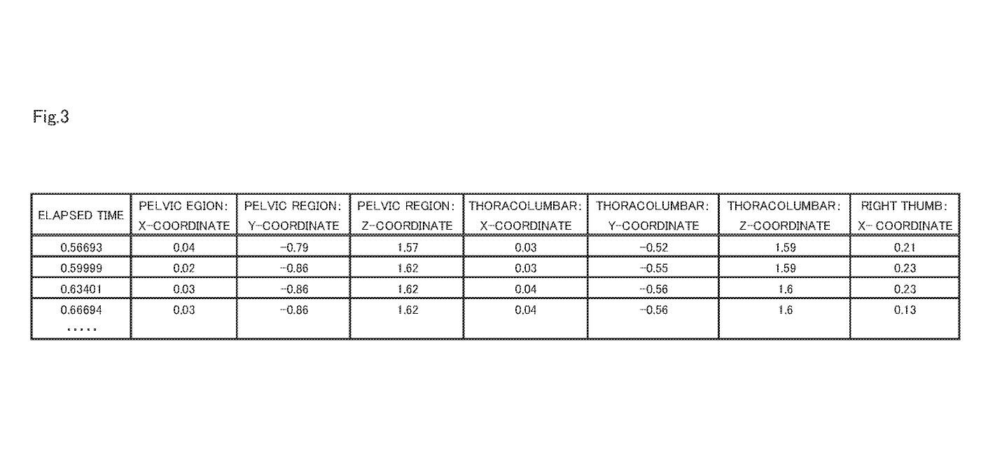

FIG. 3 is a diagram showing an example of skeleton information created in an embodiment of the present invention. As shown in FIG. 3, the skeleton information is constituted by three-dimensional coordinates of each joint at elapsed times from the start of image capture. Note that in this specification, the X-coordinate represents a value at a position in a horizontal direction on the image data, and the Y-coordinate represents a value at a position in a vertical direction on the image data, and the Z-coordinate represents a value of the depth added to a pixel.

Examples of the specific sites include the head, neck, right shoulder, right elbow, right wrist, right hand, right thumb, right fingers, left shoulder, left elbow, left wrist, left hand, left thumb, left fingers, chest, thoracolumbar, pelvic region, right hip joint, right knee, right ankle, right leg, left hip joint, left knee, left ankle, and left leg, and the like. FIG. 3 shows three-dimensional coordinates of the pelvic region, thoracolumbar, and right thumb.

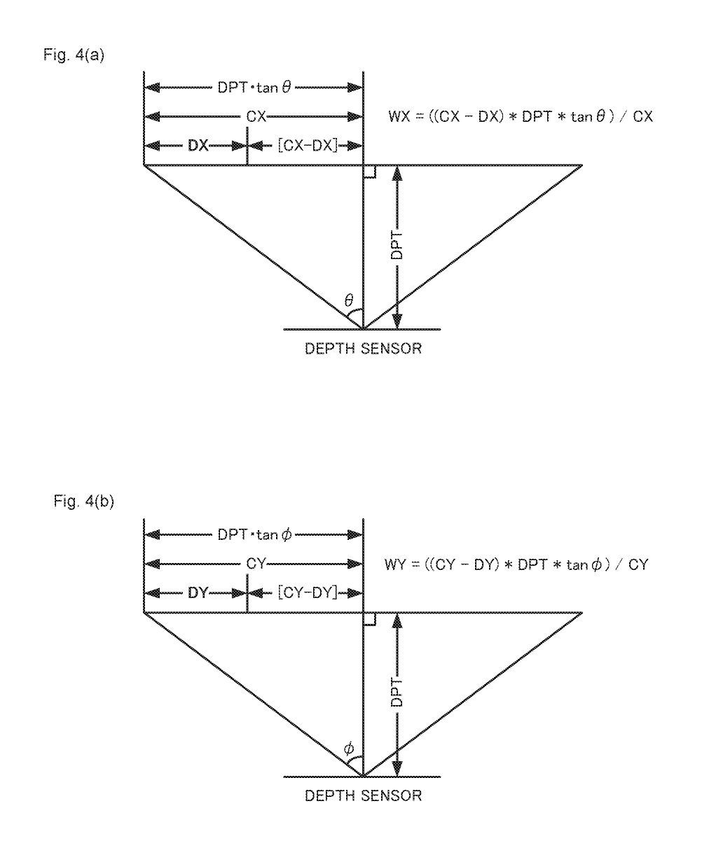

Also, a method for calculating three-dimensional coordinates from the coordinates and the depth on the image data is as follows. FIG. 4 are diagrams illustrating three-dimensional coordinate calculation processing in an embodiment of the present invention, with FIG. 4(a) showing calculation processing in the horizontal direction (X-coordinate) of an image and FIG. 4(b) showing calculation processing in the vertical direction (Y-coordinate) of the image.

First, the coordinates of a specific point are (DX, DY) and the depth at the specific point is DPT on the image data to which the depth has been added. Also, the number of pixels in the horizontal direction on the image data is 2CX, and the number of pixels in the vertical direction is 2CY. The viewing angle in the horizontal direction of the depth sensor is 20, and the viewing angle in the vertical direction is ap. In this case, as is understood from FIGS. 4(a) and 4(b), three-dimensional coordinates (WX, WY, WZ) of the specific point are calculated by Equations 1 to 3 below.

WX=((CX−DX)×DPT×tan θ)/CX Equation 1

WY=((CY−DY)×DPT×tan φ)/CY Equation 2

WZ=DPT Equation 3

Also, in the present embodiment, the state specifying unit 13 specifies the position of each site of the subject 40 from the skeleton information, and determines, from the specified position of each site, which predetermined patterns the back, the upper limbs, and the lower limbs respectively correspond to. The states of the back, the upper limbs, and the lower limbs are specified from the result of this determination.

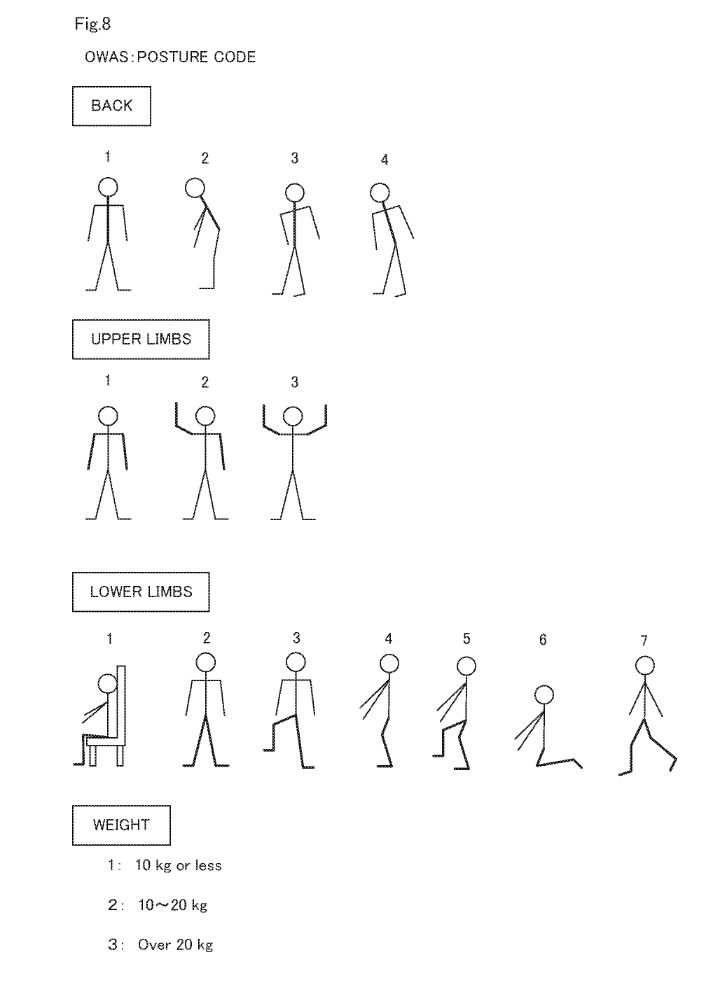

Specifically, the state specifying unit 13 determines which posture code shown in FIG. 8 the posture of the subject 40 corresponds to, for each of the back, the upper limbs, and the lower limb, using the three-dimensional coordinates of each site that are specified from the skeleton information.

Also, at this time, the state specifying unit 13 selects a site (for example, right leg, left leg) that is located at a position that is closest to a contact area from among the sites of the left and right lower limbs whose positions are specified, and detects the position (Y-coordinate) of the contact area of the subject 40 using the position of the selected site (Y-coordinate). The state specifying unit 13 then determines a pattern (posture code) for the lower limbs using the detected position of the contact area as a reference.

For example, the state specifying unit 13 compares the position of the contact area to the positions of the right leg and the left leg of the subject 40, and determines whether the lower limbs of the subject 40 correspond to “Weight on one leg” (lower limb code 3) or “Semi-crouching with weight on one leg” (lower limb code 5) (see FIG. 8). Also, the state specifying unit 13 compares the position of the contact area to the positions of the right knee and the left knee of the subject 40, and determines whether the lower limbs of the subject 40 correspond to “Kneeling on both knees” or “Kneeling on one knee” (lower limb code 6).

In the present embodiment, the posture analysis unit 14 determines whether the posture of the subject 40 has a risk by collating patterns of the back, the upper limbs, and the lower limbs that were determined with a risk table in which the relationship between the patterns and risks is predetermined.

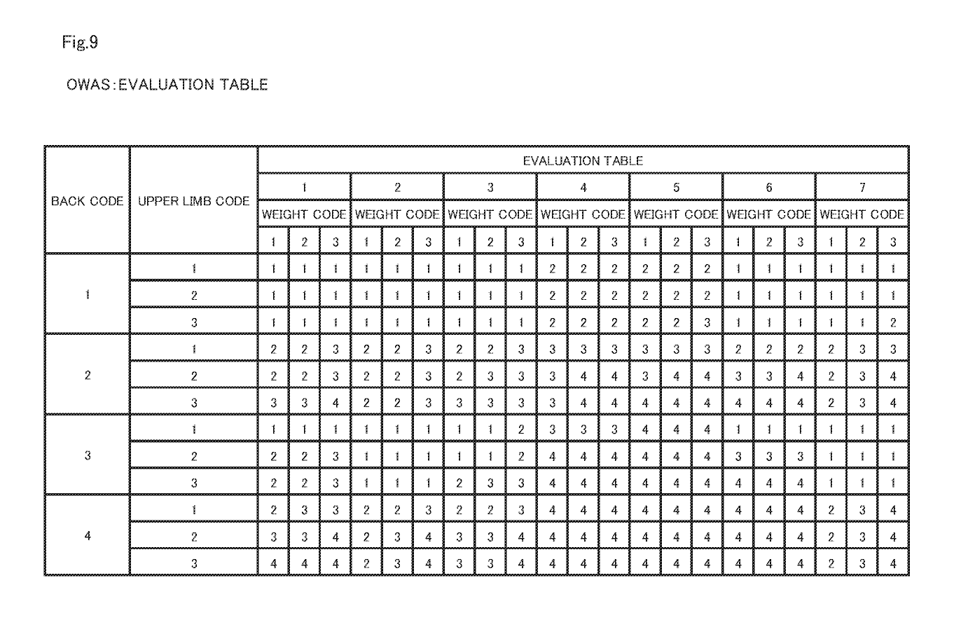

Specifically, the posture analysis unit 14 collates the codes of the back, the upper limbs, and the lower limbs that were determined by the state specifying unit 13 with the evaluation table shown in FIG. 9, and specifies the corresponding risk. The posture analysis unit 14 then notifies the terminal device 30 of the codes determined by the state specifying unit 13 and the specified risk to. Accordingly, the notified content is displayed on a screen of the terminal device 30.

Device Operations

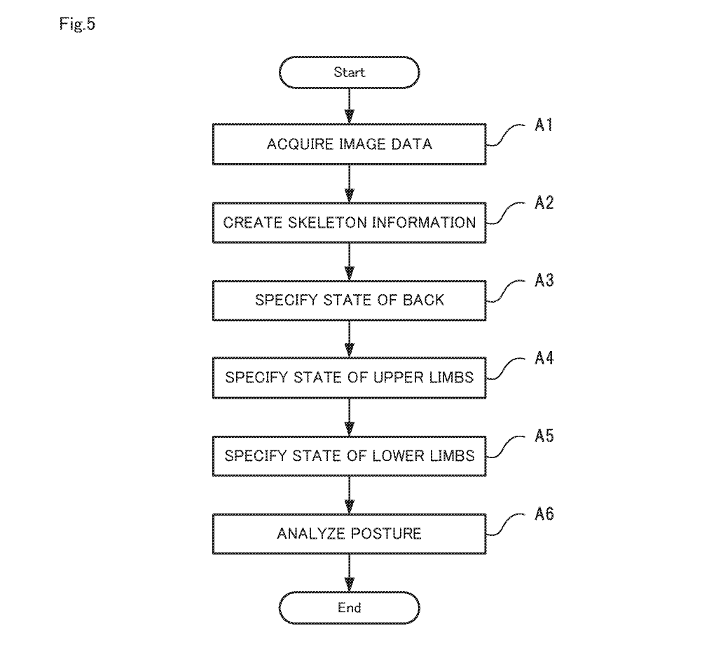

Next, operations of the posture analysis device 10 in an embodiment of the present invention will be described with reference FIG. 5. FIG. 5 is a flowchart showing operations of the posture analysis device in the embodiment of the present invention. In the following description, FIGS. 1 to 4 will be referred to as appropriate. Also, in the present embodiment, a posture analysis method is implemented by operating the posture analysis device 10. Thus, a description of the posture analysis method in the present embodiment will be replaced with the following description of the operations of the posture analysis device 10.

As shown in FIG. 5, first, the data acquisition unit 11 acquires image data with the depth that was output from the depth sensor 20 (step A1).

Next, the skeleton information creation unit 12 creates skeleton information for specifying positions of a plurality of sites of the subject 40 based on the image data acquired in step A1 (step A2).

Next, the state specifying unit 13 specifies the state of the back of the subject 40 based on the skeleton information created in step A2 (step A3). Specifically, the state specifying unit 13 acquires three-dimensional coordinates of the head, neck, chest, thoracolumbar, and pelvic region from the skeleton information, and determines which back code shown in FIG. 8 the back of the subject 40 corresponds to, using the acquired three-dimensional coordinates.

Next, the state specifying unit 13 specifies the state of the upper limbs of the subject 40 based on the skeleton information created in step A2 (step A4). Specifically, the state specifying unit 13 acquires three-dimensional coordinates of the right shoulder, right elbow, right wrist, right hand, right thumb, right fingers, left shoulder, left elbow, left wrist, left hand, left thumb, and left fingers from the skeleton information, and determines which upper limb code shown in FIG. 8 the upper limbs of the subject 40 correspond to, using the acquired three-dimensional coordinates.

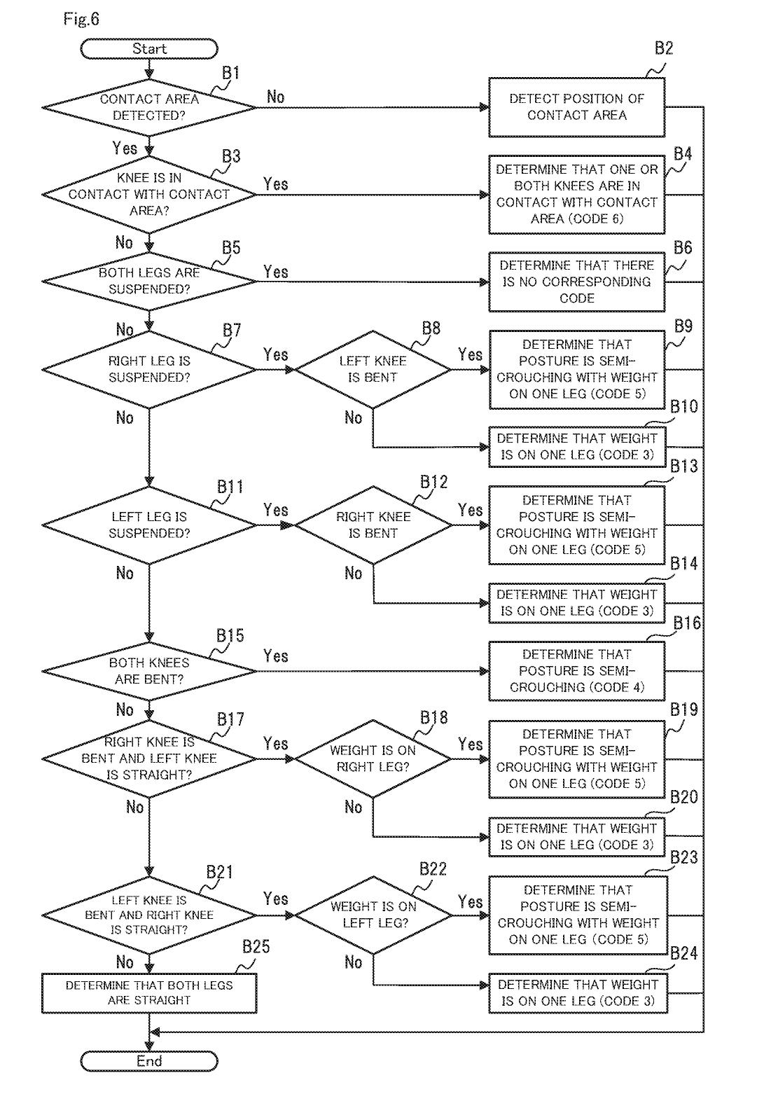

Next, the state specifying unit 13 specifies the state of the lower limbs of the subject 40 based on the skeleton information created in step A2 (step A5). Specifically, the state specifying unit 13 acquires three-dimensional coordinates of the right hip joint, right knee, right ankle, right leg, left hip joint, left knee, left ankle, and left leg from the skeleton information, and determines which lower limb code shown in FIG. 8 the lower limbs of the subject 40 correspond to, using the acquired three-dimensional coordinates. Note that step A5 will be described more specifically with reference to FIG. 6.

Next, the posture analysis unit 14 analyzes the posture of the subject 40 based on the states of the back, the upper limbs, and the lower limbs of the subject 40 (step A6). Specifically, the posture analysis unit 14 collates the codes of the back, the upper limbs, and the lower limbs that were determined by the state specifying unit 13 with the evaluation table shown in FIG. 9, and specifies the corresponding risk. The posture analysis unit 14 then notifies the terminal device 30 of the codes determined by the state specifying unit 13 and the specified risk. Note that it is assumed that the analyst has set the weight code in advance.

Because the determined codes and the specified risk are displayed on the screen of the terminal device 30 by execution of steps A1 to A6 above, the analyst can predict a health disorder occurrence risk of a worker or the like simply by checking the screen. Also, steps A1 to A6 are executed in a repetitive manner every time image data is output from the depth sensor 20.

Next, lower limb code determination processing (step A5) shown in FIG. 5 will be more specifically described with reference to FIG. 6. FIG. 6 is a flowchart that specifically shows the lower limb code determination processing shown in FIG. 5.

As shown in FIG. 6, first, the state specifying unit 13 determines whether the position of the contact area of the subject 40 has been detected (step B1). As a result of the determination in step B1, if the position of the contact area has not been detected, the posture analysis unit 14 executes detection of the position of the contact area (step B2).

Specifically, in step B1, the state specifying unit 13 selects a site (for example, right leg, left leg) that is located at a position that is closest to the contact area from among the sites of the left and right legs whose positions are specified, and detects the Y-coordinate of the contact area of the subject 40 using the Y-coordinate of the selected site.

Also, because the position of the contact area cannot be correctly detected when the subject 40 is jumping, the state specifying unit 13 may detect the Y-coordinate of the contact area using a plurality pieces of image data that are output during a set time period.

After execution of step B3, processing performed by the state specifying unit 13 ends. The states of the lower limbs are specified based on the image data that is output next. Note that, in order to increase the detection accuracy of the position of the contact area, the state specifying unit 13 can also periodically execute step B1.

On the other hand, as a result of the determination in step B1, if the position of the contact area has already been detected, the posture analysis unit 14 determines whether the knee of the subject 40 is in contact with the contact area (step B3).

Specifically, in step B3, the state specifying unit 13 acquires the Y-coordinate of the right knee from the skeleton information, calculates a difference between the acquired Y-coordinate of the right knee and the Y-coordinate of the contact area, and if the calculated difference is a threshold or less, determines that the right knee is in contact with the contact area. Also, similarly, the state specifying unit 13 acquires the Y-coordinate of the left knee from the skeleton information, calculates a difference between the acquired Y-coordinate of the left knee and the Y-coordinate of the contact area, and if the calculated difference is a threshold or less, determines that the left knee is in contact with the contact area.

As a result of the determination in step B3, if either the right knee or the left knee is in contact with the contact area, the state specifying unit 13 determines that the state of the lower limbs is code 6 (one or both knees are in contact with ground) (step B4).

Also, as a result of the determination in step B3, if neither knee is in contact with the contact area, the state specifying unit 13 determines whether both legs of the subject 40 are suspended above the contact area (step B5).

Specifically, in step B5, the state specifying unit 13 acquires the Y-coordinates of the right leg and the left leg from the skeleton information, calculates a difference between each of the acquired Y-coordinates and the Y-coordinate of the contact area, and if both of the calculated differences exceed the threshold, determines that both legs are suspended above the contact area.

As a result of the determination in step B5, if both legs are suspended above the contact area, the state specifying unit 13 determines that there is no corresponding code (step B6).

On the other hand, as a result of the determination in step B5, if both legs are not suspended above the contact area, the state specifying unit 13 determines whether the right leg is suspended above the contact area (step B7). Specifically, in step B7, if the difference, which was calculated in step B5, between the Y-coordinate of the right leg and the Y-coordinate of the contact area exceeds the threshold, then the state specifying unit 13 determines that the right leg is suspended above the contact area.

As a result of the determination in step B7, if the right leg is suspended above the contact area, the state specifying unit 13 further determines whether the left knee is bent (step B8).

Specifically, in step B8, the state specifying unit 13 acquires the three-dimensional coordinates of the left hip joint, left knee, and left ankle from the skeleton information, and calculates the distance between the left hip joint and the left knee and the distance between the left knee and the left ankle using the acquired three-dimensional coordinates. The state specifying unit 13 then calculates the angle of the left knee using the three-dimensional coordinates and the distances, and if the calculated angle is a threshold (for example, 150 degrees) or less, determines that the left knee is bent.

As a result of the determination in step B8, if the left knee is bent, the state specifying unit 13 determines that the state of the lower limbs is code 5 (semi-crouching with weight on one leg) (step B9). On the other hand, as a result of the determination in step B8, if the left knee is not bent, the state specifying unit 13 determines that the state of the lower limbs is code 3 (Weight on one leg) (step B10).

Also, as a result of the determination in step B7, if the right leg is not suspended above the contact area, the state specifying unit 13 determines whether the left leg is suspended above the contact area (step B11). Specifically, in step B11, if the difference, which was calculated in step B5, between the Y-coordinate of the left leg and the Y-coordinate of the contact area exceeds the threshold, then the state specifying unit 13 determines that the left leg is suspended above the contact area.

As a result of the determination in step B11, if the left leg is suspended above the contact area, the state specifying unit 13 further determines whether the right knee is bent (step B12).

Specifically, in step B12, the state specifying unit 13 acquires the three-dimensional coordinates of the right hip joint, right knee, and right ankle from the skeleton information, and calculates the distance between the right hip joint and the right knee and the distance between the right knee and the right ankle using the acquired three-dimensional coordinates. The state specifying unit 13 then calculates the angle of the right knee using the three-dimensional coordinates and the distances, and if the calculated angle is the threshold (for example, 150 degrees) or less, determines that the right knee is bent.

As a result of the determination in step B12, if the right knee is bent, the state specifying unit 13 determines that the state of the lower limbs is code 5 (semi-crouching with weight on one leg) (step B13). On the other hand, as a result of the determination in step B12, if the right knee is not bent, the state specifying unit 13 determines that the state of the lower limbs is code 3 (Weight on one leg) (step B14).

Also, as a result of the determination in step B11, if the left leg is not suspended above the contact area, the state specifying unit 13 determines whether both knees are bent (step B15).

Specifically, in step B15, similarly to step B8, the state specifying unit 13 calculates the angle of the right knee, and similarly to step B12, also calculates the angle of the left knee. If the angles of the right and left knees are each the threshold (for example, 150 degrees) or less, the state specifying unit 13 then determines that both knees are bent.

As a result of the determination in step B15, if both knees are bent, the state specifying unit 13 determines that the state of the lower limbs is code 4 (semi-crouching) (step B16). On the other hand, as a result of the determination in step B15, if both knees are not bent, the state specifying unit 13 determines whether the right knee is bent and the left knee is straight (step B17).

Specifically, in step B17, if, out of the angles of the right knee and the left knee that were calculated in step B15, only the angle of the right knee is the threshold (for example, 150 degrees) or less, the state specifying unit 13 determines that the right knee is bent and the left knee is straight.

Next, as a result of the determination in step B17, if the right knee is bent and the left knee is straight, the state specifying unit 13 determines whether the weight of the subject 40 is on the right leg (step B18).

Specifically, in step B18, the state specifying unit 13 acquires the three-dimensional coordinates of the pelvic region, the right leg, and the left leg from the skeleton information, and calculates the distance between the pelvic region and the right leg and the distance between the pelvic region and the left leg using the acquired three-dimensional coordinates. The state specifying unit 13 then compares the two calculated distances, and if the distance between the pelvic region and the left leg is larger than the distance between the pelvic region and the right leg, the state specifying unit 13 determines that the weight of the subject 40 is on the right leg.

As a result of the determination in step B18, if the weight of the subject 40 is on the right leg, the state specifying unit 13 determines that the state of the lower limbs is code 5 (Semi-crouching with weight on one leg) (step B19). On the other hand, as a result of the determination in step B18, if the weight of the subject 40 is not on the right leg, the state specifying unit 13 determines that the state of the lower limbs is code 3 (Weight on one leg) (step B20).

Also, as a result of the determination in step B17, if not in a state where the right knee is bent and the left knee is straight, the state specifying unit 13 determines whether the left knee is bent and the right knee is straight (step B21).

Specifically, in step B21, if, out of the angles of the right knee and the left knee that were calculated in step B15, only the angle of the left knee is the threshold (for example, 150 degrees) or less, the state specifying unit 13 determines that the left knee is bent, and the right knee is straight.

Next, as a result of the determination in step B21, if the left knee is bent and the right knee is straight, the state specifying unit 13 determines whether the weight of the subject 40 is on the left leg (step B22).

Specifically, in step B22, the state specifying unit 13 acquires the three-dimensional coordinates of the pelvic region, the right leg, and the left leg from the skeleton information, and calculates the distance between the pelvic region and the right leg and the distance between the pelvic region and the left leg using the acquired three-dimensional coordinates. The state specifying unit 13 then compares the two calculated distances, and if the distance between the pelvic region and the right leg is larger than the distance between the pelvic region and the left leg, the state specifying unit 13 determines that the weight of the subject 40 is on the left leg.

As a result of the determination in step B22, if the weight of the subject 40 is on the left leg, the state specifying unit 13 determines that the state of the lower limbs is code 5 (Semi-crouching with weight on one leg) (step B23). On the other hand, as a result of the determination in step B22, if the weight of the subject 40 is not on the left leg, the state specifying unit 13 determines that the state of the lower limbs is code 3

(Weight on one leg) (step B24).

Next, as a result of the determination in step B21, if not in a state where the left knee is bent and the right knee is straight, the state specifying unit 13 determines that the legs of the subject 40 are straight (step B25).

The states of the lower limbs are specified by the lower limb codes shown in FIG. 8 through steps B1 to B25 above. Note that from steps B1 to B25, although determinations are not made for codes 1 and 7, with regard to code 1, a determination can be made by disposing a pressure sensor on a chair or the like and inputting sensor data transmitted from the pressure sensor to the posture analysis device 10. Also, with regard to code 7, a determination can be made by calculating the moving speed of the pelvic region, for example.

### Effects in Embodiment

As described above, according to the present embodiment, simply by having the subject 40 perform work in front of the depth sensor 20, a code corresponding to the motion of the subject 40 is specified, and a risk of health disorders of the subject 40 can be determined without performing manual tasks.

### Variations

Although the depth sensor 20 is used in order to acquire data that varies according to a motion of the subject 40 in the above-described example, the means for acquiring data is not limited to the depth sensor 20 in the present embodiment. In the present embodiment, a motion capture system may be used, instead of the depth sensor 20. Also, the motion capture system may be any of optical, inertial sensor, mechanical, magnetic, and video type systems.

Program

A program in the present embodiment may be a program for causing a computer to execute steps A1 to A6 shown in FIG. 5. This program is installed in the computer, and executed by the computer, and thereby the posture analysis device 10 and the posture analysis method in the present embodiment can be realized. In this case, a CPU (Central Processing Unit) of the computer functions as the data acquisition unit 11, the skeleton information creation unit 12, the state specifying unit 13, and the posture analysis unit 14, and performs processing.

Also, the program in the present embodiment may be executed by a computer system constructed by a plurality of computers. In this case, each of the computers may function as any one of the data acquisition unit 11, the skeleton information creation unit 12, the state specifying unit 13, and the posture analysis unit 14, for example.

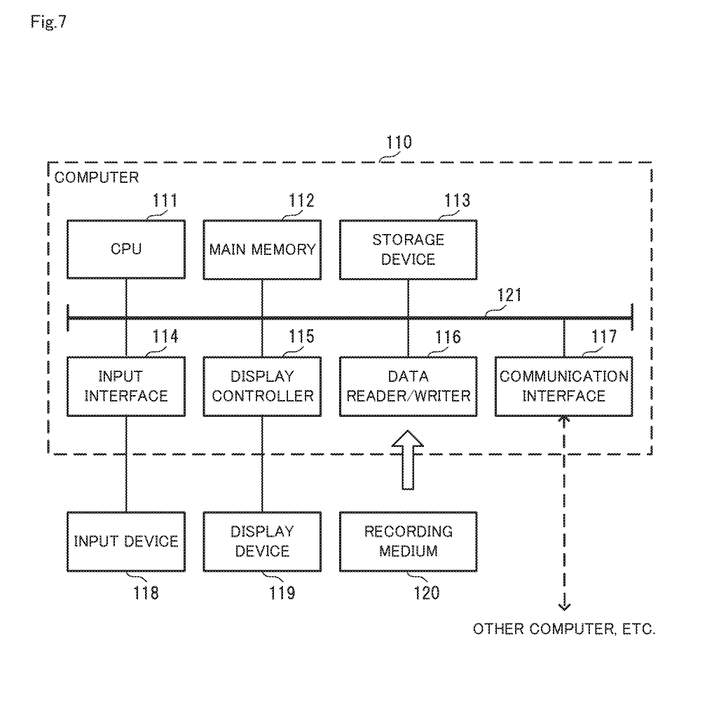

Here, a computer configured to realize the posture analysis device 10 by executing the program in the present embodiments will be described with reference to FIG. 7. FIG. 7 is a block diagram showing an example of a computer for realizing the posture analysis device in an embodiment of the present invention.

As shown in FIG. 7, the computer 110 includes a CPU 111, a main memory 112, a storage device 113, an input interface 114, a display controller 115, a data reader/writer 116, and a communication interface 117. These units are connected via a bus 121 for data communication.

The CPU 111 loads the programs (codes) stored in the storage device 113 in the present embodiment on the main memory 112, executes these programs in a predetermined order, and thereby implements various calculations. Typically, the main memory 112 is a volatile storage device such as a DRAM (Dynamic Random Access Memory). Also, the program in the present embodiment is provided in a state of being stored in a computer-readable recording medium 120. Note that the program in the present embodiment may be distributed on the Internet connected via the communication interface 117.

Also, specific examples of the storage device 113 include a semiconductor storage device such as a flash memory as well as a hard disk drive. The input interface 114 mediates data transmission between the CPU 111 and input devices 118 such as a keyboard and a mouse. The display controller 115 is connected to a display device 119, and controls the display on the display apparatus 119.

The data reader/writer 116 mediates data transmission between the CPU 111 and the recording medium 120, reads out a program from the recording medium 120, and executes writing of the result of processing by the computer 110 to the recording medium 120. The communication interface 117 mediates data transmission between the CPU 111 and another computer.

Also, specific examples of the recording medium 120 include a general-purpose semiconductor storage device such as CF (Compact Flash (registered trademark)) or SD

(Secure Digital), a magnetic recording medium such as a Flexible Disk, or an optical storage medium such as a CD-ROM (Compact Disk Read Only Memory).

Note that the posture analysis device 10 in the present embodiment can be realized by not only a computer on which programs are installed but also hardware corresponding to each unit. Furthermore, a portion of the posture analysis device 10 may be realized by a program and the remaining portion thereof may be realized by hardware.

Part or all of the above-described embodiments can be expressed by Supplementary Notes 1 to 15 below, but are not limited thereto.

Supplementary Note 1

A posture analysis device for analyzing a posture of a subject, the device including:

a data acquisition unit configured to acquire data that varies according to a motion of the subject;

a skeleton information creation unit configured to create skeleton information for specifying positions of a plurality of sites of the subject based on the data;

a state specifying unit configured to specify states of a back, upper limbs, and lower limbs of the subject based on the skeleton information; and

a posture analysis unit configured to analyze the posture of the subject based on the specified states of the back, the upper limbs, and the lower limbs of the subject.

Supplementary Note 2

The posture analysis device according to Supplementary Note 1,

in which the data acquisition unit acquires, as the data, image data to which a depth of each pixel is added, from a depth sensor that is disposed to capture an image of the subject.

Supplementary Note 3

The posture analysis device according to Supplementary Note 1 or 2,

in which the state specifying unit specifies positions of sites of the subject from the skeleton information, determines which predetermined pattern the back, the upper limbs, and the lower limbs correspond to, from the specified positions of the sites, and specifies states of the back, the upper limbs, and the lower limbs based on a result of the determination.

Supplementary Note 4

The posture analysis device according to Supplementary Note 3,

in which the posture analysis unit determines whether the posture of the subject has a risk, by collating the patterns of the back, the upper limbs, and the lower limbs that were determined with a risk table in which a relationship between patterns and risks is predetermined.

Supplementary Note 5

The posture analysis device according to Supplementary Note 3 or 4,

in which the state specifying unit selects a site that is located at a position that is closest to the ground from among the sites of the lower limbs whose positions are specified, and detects a position of a contact area of the subject using the position of the selected site, and determines a pattern of the lower limbs using the detected position of the contact area as a reference.

Supplementary Note 6

A posture analysis method for analyzing a posture of a subject, the method including:

(a) a step of acquiring data that varies according to a motion of the subject;

(b) a step of creating skeleton information for specifying positions of a plurality of sites of the subject based on the data;

(c) a step of specifying states of a back, upper limbs, and lower limbs of the subject based on the skeleton information; and

(d) a step of analyzing the posture of the subject based on the specified states of the back, the upper limbs, and the lower limbs of the subject.

Supplementary Note 7

The posture analysis method according to Supplementary Note 6,

in which in step (a) above, image data to which a depth of each pixel is added is acquired as the data from a depth sensor that is disposed to capture an image of the subject.

Supplementary Note 8

The posture analysis method according to Supplementary Note 6 or 7,

in which in step (c) above, positions of sites of the subject are specified from the skeleton information, which predetermined pattern the back, the upper limbs, and the lower limbs correspond to are determined from the specified positions of the sites, and states of the back, the upper limbs, and the lower limbs are specified based on a result of the determination.

Supplementary Note 9

The posture analysis method according to Supplementary Note 8,

in which in step (d) above, whether the posture of the subject has a risk is determined by collating the patterns of the back, the upper limbs, and the lower limbs that were determined with a risk table in which a relationship between patterns and risks is predetermined.

Supplementary Note 10

The posture analysis method according to Supplementary Note 8 or 9,

in which in step (c) above, a site that is located at a position that is closest to the ground is selected from among the sites of the lower limbs whose positions are specified, and a position of a contact area of the subject is detected using the position of the selected site, and a pattern of the lower limbs is determined using the detected position of the contact area as a reference.

Supplementary Note 11

A computer-readable recording medium in which a program for analyzing a posture of a subject by a computer is recorded, the program including commands for causing the computer to execute:

(a) a step of acquiring data that varies according to a motion of the subject;

(b) a step of creating skeleton information for specifying positions of a plurality of sites of the subject based on the data;

(c) a step of specifying states of a back, upper limbs, and lower limbs of the subject based on the skeleton information; and

(d) a step of analyzing the posture of the subject based on the specified states of the back, the upper limbs, and the lower limbs of the subject.

Supplementary Note 12

The computer-readable recording medium according to Supplementary Note 11,

in which in step (a) above, image data to which a depth of each pixel is added is acquired as the data from a depth sensor that is disposed to capture an image of the subject.

Supplementary Note 13

The computer-readable recording medium according to Supplementary Note 11 or 12,

in which in step (c) above, positions of sites of the subject are specified from the skeleton information, which predetermined pattern the back, the upper limbs, and the lower limbs correspond to are determined from the specified positions of the sites, and states of the back, the upper limbs, and the lower limbs are specified based on a result of the determination.

Supplementary Note 14

The computer-readable recording medium according to Supplementary Note 13,

in which in step (d) above, whether the posture of the subject has a risk is determined by collating the patterns of the back, the upper limbs, and the lower limbs that were determined with a risk table in which a relationship between patterns and risks is predetermined.

Supplementary Note 15

The computer-readable recording medium according to Supplementary Note 13 or 14,

in which in step (c) above, a site that is located at a position that is closest to the ground is selected from among the sites of the lower limbs whose positions are specified, and a position of a contact area of the subject is detected using the position of the selected site, and a pattern of the lower limbs is determined using the detected position of the contact area as a reference.

Although the present invention has been described with reference to the embodiments, the present invention is not limited to the above-described embodiments. Various modifications that can be understood by those skilled in the art can be made to the configuration and details of the present invention within the scope of the invention.

This application claims priority to Japanese Patent Application No. 2016-124876 filed Jun. 23, 2016, the entire contents of which are incorporated herein by reference.

### INDUSTRIAL APPLICABILITY

As described above, according to the present invention, it is possible to analyze the posture of a subject without performing manual tasks. The present invention is useful at production sites, construction sites, medical sites, nursing facilities, and the like.

### REFERENCE NUMERALS

10 Posture analysis device
11 Data acquisition unit
12 Skeleton information creation unit
13 State specifying unit
14 Posture analysis unit
20 Depth sensor
30 Terminal device
40 Subject
110 Computer
111 CPU
112 Main memory
113 Storage device
114 Input interface
115 Display controller
116 Data reader/writer
117 Communication interface
118 Input device
119 Display device
120 Recording medium
121 Bus

What is claimed is:

1. A posture analysis device for analyzing a posture of a subject, the device comprising:
a data acquisition unit configured to acquire data that varies according to a motion of the subject;
a skeleton information creation unit configured to create skeleton information for specifying positions of a plurality of sites of the subject based on the data;
a state specifying unit configured to specify states of a back, upper limbs, and lower limbs of the subject based on the skeleton information; and
a posture analysis unit configured to analyze the posture of the subject based on the specified states of the back, the upper limbs, and the lower limbs of the subject.

2. The posture analysis device according to claim 1,
wherein the data acquisition unit acquires, as the data, image data to which a depth of each pixel is added, from a depth sensor that is disposed to capture an image of the subject.

3. The posture analysis device according to claim 1,
wherein the state specifying unit specifies positions of sites of the subject from the skeleton information, determines which predetermined pattern the back, the upper limbs, and the lower limbs correspond to, from the specified positions of the sites, and specifies states of the back, the upper limbs, and the lower limbs based on a result of the determination.

4. The posture analysis device according to claim 3,
wherein the posture analysis unit determines whether the posture of the subject has a risk, by collating the patterns of the back, the upper limbs, and the lower limbs that were determined with a risk table in which a relationship between patterns and risks is predetermined.

5. The posture analysis device according to claim 3,
wherein the state specifying unit selects a site that is located at a position that is closest to the ground from among the sites of the lower limbs whose positions are specified, and detects a position of a contact area of the subject using the position of the selected site, and determines a pattern of the lower limbs using the detected position of the contact area as a reference.

6. A posture analysis method for analyzing a posture of a subject, the method comprising:
(a) a step of acquiring data that varies according to a motion of the subject;
(b) a step of creating skeleton information for specifying positions of a plurality of sites of the subject based on the data;
(c) a step of specifying states of a back, upper limbs, and lower limbs of the subject based on the skeleton information; and
(d) a step of analyzing the posture of the subject based on the specified states of the back, the upper limbs, and the lower limbs of the subject.

7. The posture analysis method according to claim 6,
wherein in step (a) above, image data to which a depth of each pixel is added is acquired as the data from a depth sensor that is disposed to capture an image of the subject.

8. The posture analysis method according to claim 6,
wherein in step (c) above, positions of sites of the subject are specified from the skeleton information, which predetermined pattern the back, the upper limbs, and the lower limbs correspond to are determined from the specified positions of the sites, and states of the back, the upper limbs, and the lower limbs are specified based on a result of the determination.

9. The posture analysis method according to claim 8,
wherein in step (d) above, whether the posture of the subject has a risk is determined by collating the patterns of the back, the upper limbs, and the lower limbs that were determined with a risk table in which a relationship between patterns and risks is predetermined.

10. The posture analysis method according to claim 8,
wherein in step (c) above, a site that is located at a position that is closest to the ground is selected from among the sites of the lower limbs whose positions are specified, and a position of a contact area of the subject is detected using the position of the selected site, and a pattern of the lower limbs is determined using the detected position of the contact area as a reference.

11. A non-transitory computer-readable recording medium in which a program for analyzing a posture of a subject by a computer is recorded, the program including commands for causing the computer to execute:
(a) a step of acquiring data that varies according to a motion of the subject;
(b) a step of creating skeleton information for specifying positions of a plurality of sites of the subject based on the data;
(c) a step of specifying states of a back, upper limbs, and lower limbs of the subject based on the skeleton information; and
(d) a step of analyzing the posture of the subject based on the specified states of the back, the upper limbs, and the lower limbs of the subject.

12. The non-transitory computer-readable recording medium according to claim 11,
wherein in step (a) above, image data to which a depth of each pixel is added is acquired as the data from a depth sensor that is disposed to capture an image of the subject.

13. The non-transitory computer-readable recording medium according to claim 11,
wherein in step (c) above, positions of sites of the subject are specified from the skeleton information, which predetermined pattern the back, the upper limbs, and the lower limbs correspond to are determined from the specified positions of the sites, and states of the back, the upper limbs, and the lower limbs are specified based on a result of the determination.

14. The non-transitory computer-readable recording medium according to claim 13,
wherein in step (d) above, whether the posture of the subject has a risk is determined by collating the patterns of the back, the upper limbs, and the lower limbs that were determined with a risk table in which a relationship between patterns and risks is predetermined.

15. The non-transitory computer-readable recording medium according to claim 13,
wherein in step (c) above, a site that is located at a position that is closest to the ground is selected from among the sites of the lower limbs whose positions are specified, and a position of a contact area of the subject is detected using the position of the selected site, and a pattern of the lower limbs is determined using the detected position of the contact area as a reference.
